(19) United States
(12) Patent Application Publication
(10) Pub. No.: US 2007/0260123 A1
(43) Pub. Date: Nov. 8, 2007

(54) Apparatus and method for stabilizing body tissue

(75) Inventors: Amir K. Durrani, Cleveland Heights, OH (US); Lucas J. Burton, Orleans, IN (US); Benjamin D. Hoagland, Birmingham, AL (US); Santosh K. Tumkur, Cincinnatio, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/800,504

(22) Filed: May 4, 2007

Related U.S. Application Data
(60) Provisional application No. 60/798,808, filed on May 5, 2006.

(51) Int. Cl. A61B 1/32

(52) U.S. Cl. 600/205

(57) ABSTRACT

An apparatus for holding a body tissue in a desired orientation includes a first leg and a second leg. The first leg has longitudinally spaced distal and proximal ends. The second leg is laterally spaced from the first leg, is adapted for lateral motion with respect to the first leg, and has longitudinally spaced distal and proximal ends. The distal ends of the first and second legs are for contacting the body tissue. The first leg includes a first guide post extending toward the second leg. The second leg includes a second guide post extending toward the first leg. The first and second guide posts telescopically engage to form a guide track. A first threaded post extends from the first leg toward the second leg, and a second threaded post extends from the second leg toward the first leg. A turnbuckle body connects the first and second threaded posts to form a turnbuckle assembly. The proximal ends of the first and second legs are movably connected by the guide track and the turnbuckle assembly. The distal ends of the first and second legs each include a suction pad adapted to apply suction pressure to the body tissue to hold the body tissue in the desired orientation. A method of holding a body tissue in a desired orientation is also described.

### RELATED APPLICATION

This application claims priority from U.S. Provisional Application No. 60/798,808, filed May 5, 2006, the subject matter of which is incorporated herein by reference.

### TECHNICAL FIELD

The present invention relates to an apparatus and method for use of a stabilization device and, more particularly, to a stabilization device with laterally movable legs having a plurality of suction ports.

### BACKGROUND OF THE INVENTION

Surgeries to treat disease in the heart, particularly blockages in coronary vessels, are becoming increasingly common to treat atherosclerosis and other conditions causing reduced blood flow to the heart. For many years, surgeons have performed “open-heart” surgery to repair defects in the heart and the associated cardiovascular system. As these procedures have become more common and more costly, a need has developed for techniques to make cardiac surgical procedures less traumatic to the patient. The use of a cardiopulmonary bypass (CPB) apparatus is a primary contribution to the trauma inherent in traditional procedures. To attempt to alleviate the trauma and side effects of CPB, surgeons have begun performing cardiac surgeries without stopping the heart. To successfully perform such surgery, several challenges must be met. One particular problem confronting the surgeon who operates on the beating heart is the difficulty in performing extremely delicate surgical procedures while the contractions of the heart muscles cause the surface of the heart to continuously move.

To attempt to restrict the motion of heart at the particular surgical site where the surgeon is working, the surgeon may pass at least a pair of sutures through the exterior tissue layers of the heart. By pulling the sutures in opposite directions, the tissue is stretched, and the motion caused by the contractions of the heart muscles is reduced or partially compensated. This technique is not completely effective in preventing the natural motion of the heart and requires extra time to place the sutures, and, additionally, may cause damage to the cardiac tissue when the sutures are placed or manipulated. Preferably, the surgeon would be able to fix the motion of the cardiac tissue containing or adjacent to an area where surgery is to be performed without the need to attach or manipulate additional sutures. The ability to fix the position of the cardiac tissue in a region of the heart would permit the surgeon to perform delicate surgical procedures on the beating heart while the portion of the heart on which the surgery is performed remains substantially motionless throughout the procedure.

Several stabilization devices have been proposed to supplant the sutures and stabilize the motion of the heart tissue in a less invasive manner, mainly using various vacuum-powered systems. An example of a stabilization device is disclosed in U.S. Pat. No. 6,602,183, issued Aug. 5, 2003 to Levi et al. (hereafter referenced as the '183 patent). The '183 patent discloses a stabilization device having a plurality of suction ports adapted to engage the heart surface. At least one vacuum line connects one or more suction ports to a vacuum source through vacuum routing channels in the stabilization device and a vacuum control unit. The relative vacuum strengths applied to the heart can be adjusted to a minimum needed to hold the heart tissue in place, in order to avoid suction damage to the heart tissue. Several depicted embodiments of the device of the '183 patent include parallel legs within which the structure of the suction ports and corresponding vacuum routing channels are formed. These legs at least partially frame the surgery site on the heart surface.

The device of the '183 patent, however, does not provide ideal heart tissue stabilization. First, when multiple suction ports are fed from the same vacuum line, loss of contact/sealing with the heart tissue of one port causes almost all of the suction pressure in the vacuum feed line to be directed to that disconnected port and subsequently tissue held by other ports on the same vacuum feed line is released. Second, the complicated port/line structure within the legs and body of the device of the '183 patent is expensive and time-consuming to manufacture, due to the precision machining needed within a very small space. Third, the vacuum control unit adds complexity and may require equipment not normally provided in operating rooms. Finally, the device of the '183 patent has a fixed structure. Therefore, when the legs are placed on opposite sides of the surgery site, they cannot be relatively moved laterally to “stretch” or smooth out the tissue held therebetween, as is possible with the known suture stabilization method, and the user must manually tension the heart tissue before placing the device of the '183 patent.

Another example of a stabilization device is disclosed in U.S. Pat. No. 6,406,424, issued Jun. 18, 2002 to Williamson et al. (hereafter referenced as the '424 patent). The '424 patent discloses a stabilization device, similar to that of the '183 patent, having a plurality of suction ports formed within the structure of each of two parallel legs. As shown best in FIG. 7, the device of the '424 patent includes a turnbuckle mechanism with a threaded rod attached to each leg and a knurled wheel located between the legs and adapted to turn the rods to move the legs laterally. This motion will spread or smooth the heart tissue held by the legs, and allow for more controllable stabilization than the device of the '183 patent.

The device of the '424 patent, however, has the aforementioned problems of loss of suction from linked suction ports when one of the ports dislodges and of difficulty and expense in manufacturing the integral ports/lines in the legs, as with the device of the '183 patent. In addition, the turnbuckle structure of the device of the '424 patent includes rods (elements 333 and 334 in FIG. 7) which protrude from either side of the device and may contact the adjacent heart tissue in an unwanted manner or provide a lever point for an unintended contact by the user to lift the legs, thus dislodging the suction ports. Moreover, the knurled wheel (element 332 in FIG. 7) is laterally narrow, and thus does not contact and anchor a significant portion of the rods to resist forces applied in a normal direction to the heart surface. When these normal forces are exerted on the device of the '424 patent, the knurled wheel may bind on the rod threads and/or the device may flex in an undesirable manner. Finally, the knurled wheel of the '424 patent is recessed between two shoulders (elements 336 and 338 in FIG. 7), so a surgeon may have difficulty rotating the knurled wheel without also applying unwanted force to the adjacent shoulders, which could result in dislodgement of the device from the heart tissue.

Accordingly, it is desirable to provide a method and apparatus of a stabilization device which: resists destabilizing forces produced by a beating heart in multiple orientations, may be easily adjusted by a user, avoids unwanted dislodgement from the heart surface, uses existing vacuum sources, may be used in a timely and efficient manner, and is economical to manufacture and use.

### SUMMARY OF THE INVENTION

In an embodiment of the present invention, an apparatus for holding a body tissue in a desired orientation is described. The apparatus includes a first leg and a second leg. The first leg has longitudinally spaced distal and proximal ends. The second leg is laterally spaced from the first leg, is adapted for lateral motion with respect to the first leg, and has longitudinally spaced distal and proximal ends. The distal ends of the first and second legs are for contacting the body tissue. The first leg includes a first guide post extending toward the second leg. The second leg includes a second guide post extending toward the first leg. The first and second guide posts telescopically engage to form a guide track. A first threaded post extends from the first leg toward the second leg, and a second threaded post extends from the second leg toward the first leg. A turnbuckle body connects the first and second threaded posts to form a turnbuckle assembly. The proximal ends of the first and second legs are movably connected by the guide track and the turnbuckle assembly. The distal ends of the first and second legs each include a suction pad adapted to apply suction pressure to the body tissue to hold the body tissue in the desired orientation.

In an embodiment of the present invention, a method of holding a body tissue in a desired orientation is described. A stabilization device is provided, including a first leg having longitudinally spaced distal and proximal ends and a second leg, laterally spaced from the first leg, adapted for lateral motion with respect to the first leg and having longitudinally spaced distal and proximal ends. The proximal ends of the first and second legs are movably connected. Each of the distal ends of the first and second legs is provided with a suction pad adapted to apply suction pressure to the body tissue and including a plurality of suction ports. Each of the plurality of suction ports is placed into separate fluid communication with a vacuum source. The stabilization device is supported with a stabilizing arm. The body tissue is contacted with at least one suction pad. The body tissue is held in a desired orientation with the stabilizing arm through suction provided from the vacuum source individually to each of the plurality of suction ports.

### BRIEF DESCRIPTION OF THE DRAWINGS

For a better understanding of the invention, reference may be made to the accompanying drawings, in which:

FIG. 1 is a partial perspective front view of an embodiment of the present invention;

FIG. 2 is a partial perspective bottom view of an embodiment of the present invention;

FIG. 3 is a partial perspective front view, similar to that of FIG. 1, of an embodiment of the present invention;

FIG. 4A is a top view of an embodiment of the present invention in a first mode; and

FIG. 4B is a top view of an embodiment of the present invention in a second mode.

### DESCRIPTION OF EMBODIMENTS

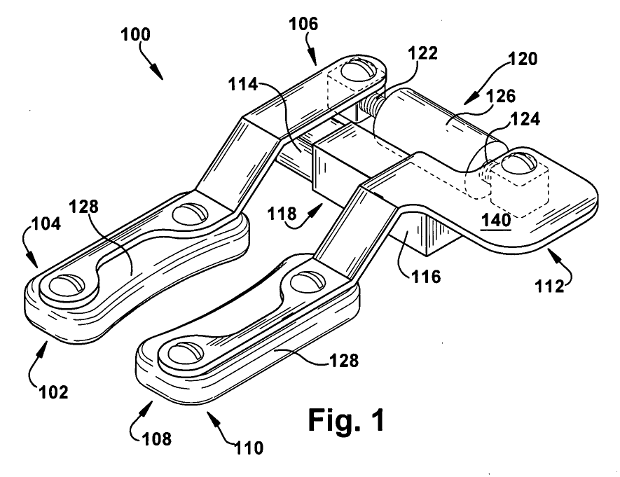

In accordance with the present invention, FIG. 1 depicts a stabilization device 100 for holding a body tissue in a desired orientation. Though the following description presumes that the stabilization device 100 holds a heart tissue steady during a cardiac surgical procedure, the stabilization device may be used in any suitable application, to hold any body tissue in any desired orientation.

The stabilization device 100 includes a first leg 102 having longitudinally spaced distal and proximal ends 104 and 106, respectively, and a second leg 108, laterally spaced from the first leg and having longitudinally spaced distal and proximal ends 110 and 112, respectively. The distal ends 104 and 110 are adapted to contact the heart tissue. The first and second legs 102 and 108 may be made of any suitable material but should be sufficiently rigid to impose the desired stabilizing force on the heart tissue. The first and second legs 102 and 108 may be parallel and lie in substantially the same plane, as shown in FIGS. 1-4B, or may have any other desired angular orientation to each other.

The first leg 102 includes a first guide post 114 extending toward the second leg 108, and the second leg includes a second guide post 116 extending toward the first leg. The first and second guide posts 114 and 116, respectively, telescopically engage to form a guide track 118. The first and second guide posts 114 and 116 may be of any suitable structure, cross-sectional shape, or construction. For example, a hollow cross-sectional structure may reduce weight of the first and second guide posts 114 and 116 while maintaining resistance to bending stress. The first and second guide posts 114 and 116 need not match in any respect except as useful for the described telescopic compressing or condensing engagement. The first and second guide posts 114 and 116 may have a telescoping engagement wherein at least a portion of one of the first and second guide posts is contained within the other of the first and second guide posts. Alternately, a clip or clamp (not shown) could slidably connect first and second guide posts 114 and 116 extending parallel beside one another for a suitably telescoping engagement of the first and second guide posts.

The stabilization device 100 according to the present invention also includes a turnbuckle assembly 120. A first threaded post 122 extends from the first leg 102 toward the second leg 108, and a second threaded post 124 extends from the second leg toward the first leg. A turnbuckle body 126 engages the first and second threaded posts 122 and 124, respectively, (shown partially in phantom in FIG. 1) to form the turnbuckle assembly 120. The turnbuckle body 126 may be smaller in a longitudinal dimension than in a lateral dimension, as is the elongated cylindrical structure shown in FIG. 1, as desired for compactness and ease of operation. The turnbuckle body 126 may be made of, or surfaced with, one or more materials having frictional properties chosen to facilitate intentional rotation of the turnbuckle body by the user while resisting unintentional rotation of the turnbuckle body by lateral forces transmitted through the first and second threaded posts 122 and 124.

As shown in FIG. 1, the proximal ends 106 and 112 of the first and second legs 102 and 104 may be vertically offset from the distal ends 104 and 110. This offset prevents the turnbuckle assembly 120 and guide track 118 from contacting and possibly pinching the heart surface. Additionally, the offset “plafform” formed by the proximal ends 106 and 112 of the first and second legs 102 and 104 may provide the user with a finger rest above the heart tissue, as well as serving as a stable point of reference with respect to the heart tissue to assist the user's depth perception in overcoming a common optical illusion caused by slight motion of the heart tissue at the surgical site.

The distal ends 104 and 110 of the first and second legs 102 and 108, respectively, each include at least one suction pad 128 adapted to apply suction pressure to engage the body tissue being stabilized by the stabilization device 100. The suction pads 128 are shown in FIG. 1 as each being a resilient element fastened to a rigid portion of the first and second legs 102 and 108, with mirror-image structures which are contoured to provide a wide surgical site between the first and second legs. The suction pads 128, however, may be of any suitable resilient or rigid material or structure and may readily be chosen for a particular application of the present invention by one of ordinary skill in the art.

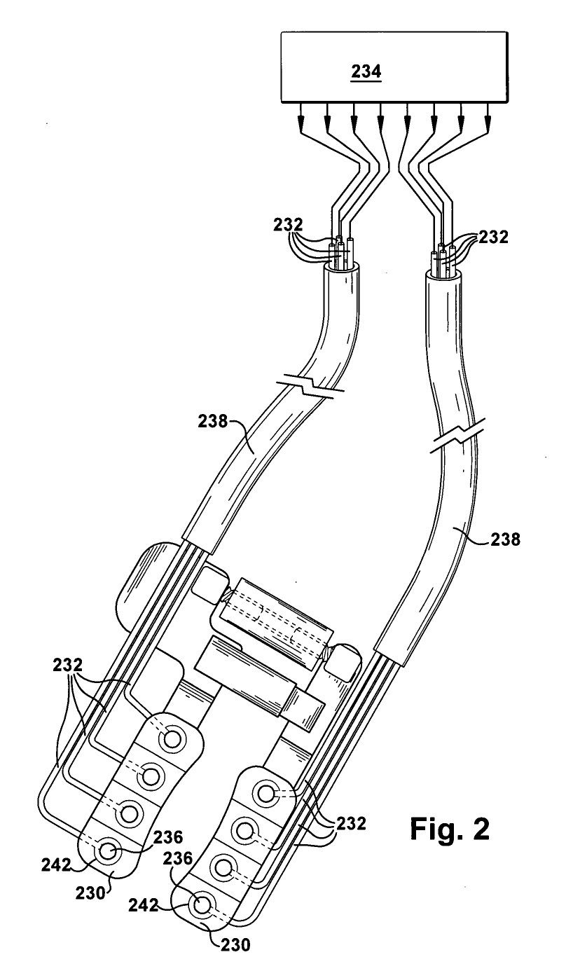

Each suction pad 128 may include one or more suction ports 230, shown in FIG. 2 as four suction ports 230 per suction pad 128. The suction ports 230 are structured such that, when the suction pad 128 is brought into contact with the heart tissue, each suction port is fluidly separated from the other suction ports. A plurality of suction tubes 232 connect the suction ports 230 with a vacuum source 234, optionally through a suction channel 236, shown partially in phantom in FIG. 2. The suction channels 236 may be machined or molded into the suction pads 128, and optionally have a simple structure, such as the depicted inverted L-shaped hole configured to accept a suction tube 232 in a frictional fit and to place the suction tube into fluid communication with the corresponding suction port 230. The location and configuration of the suction channels 236 may be chosen to provide the shortest practicable connection between each suction port 230 and the suction tube 232.

As shown in FIG. 2, each suction tube 232 may connect one suction port 230 with the vacuum source 234, so that each suction port is in separate fluid communication with the vacuum source and dislodgement of one suction port does not cause a loss of suction in the other suction ports of the same suction pad 128. However, a single suction tube 232 could connect two or more suction ports 230 with the vacuum source 234, as desired. In this arrangement, each suction tube 232 may provide independent and separate suction control to its associated suction port(s) 230.

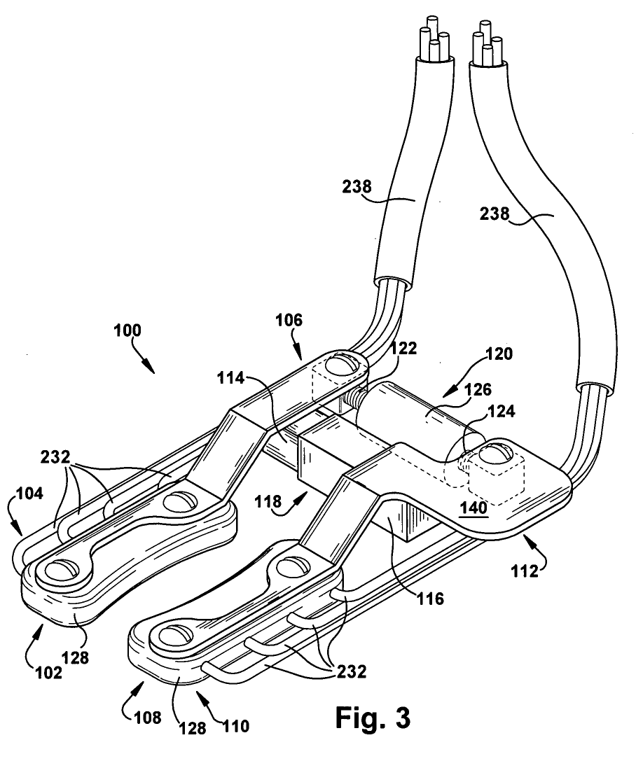

A plurality of suction tubes 232 may be bundled together by an outer sheath 238 along at least a portion of the length of the suction tubes 232, for ease in handling during use. FIG. 3 depicts a top view of a stabilization device 100 according to the present invention with the suction tubes 232 routed under the proximal ends 106 and 112 of the first and second legs 102 and 108, to allow the user easy access to the turnbuckle body 126. However, any suitable routing of the suction tubes 232 or bundling with outer sheaths 238, tie wraps/cable ties (not shown), or any other containment or support structure may be provided to the stabilization device 100.

As shown schematically in FIG. 2, the suction tubes 232 may each be individually directly attached to the vacuum source 234. Alternately, two or more of the suction tubes 232 may be connected with a larger “trunk” vacuum feed line (not shown) of any desired length, with the trunk line being attached to the vacuum source 234. In the latter case, one or more trunk lines may provide suction from the vacuum source 234 to a single stabilization device 100. The trunk lines or another suitable adapter may be provided when the suction tubes 232 are of a different configuration or size than the vacuum outlet ports of a standard operating room vacuum source 234.

Each suction tube 232 may be dimensioned in length, diameter, or both to deliver a desired suction pressure from the vacuum source 234 to the suction port(s) 230 associated with that suction tube. For example, a longer suction tube 232 may result in a lower suction pressure ultimately delivered to the associated suction port(s) 230. In such a manner, a “test port” could be provided, with which the vacuum pressure provided to the stabilization device 100 is gradually reduced until loss of heart tissue engagement at the test port indicates that the vacuum level of the remaining suction ports 230 is at a minimum needed to stabilize the heart tissue.

One or both of the first and second legs 102 and 108 may include a mounting plate 140, as shown extending from the second leg 108 in FIG. 1, adapted to engage with a stabilizing arm (not shown) and hold the body tissue in a desired orientation with respect to the stabilizing arm. For example, the mounting plate 140 may be grasped by a clamp attached to the stabilizing arm. The mounting plate 140 may also or instead include additional structures (not shown) as needed to engage a stabilizing arm in a desired manner.

One more of the suction ports 230 may include a suction cup 242, as shown in FIG. 2, to assist in holding the body tissue. The suction cups 242, when present, may be of a more flexible resilient material than that of the suction ports 230 so that the suction cups can adjust or flex within their respective suction ports to help each individual suction port retain engagement with moving body tissue. The suction tubes 232 may connect directly to the suction cups 242 inside the suction ports 230 and thereby provide suction pressure to the concave interior portions of the suction cups 242.

When the suction tubes 232 connect to the suction cups 242 directly, the suction cups may protrude slightly below the suction ports 230 to provide desirable contact between the suction cups and the body tissue while the more-rigid suction ports 230 structurally stabilize the suction cups against lateral movement. Alternately, the suction tubes 232 could connect to the suction ports 230 and provide suction pressure to a region of the body tissue held within the suction ports but outside the suction cups 242 to provide a redundant source of suction pressure to hold the body tissue. In the latter case, the suction cups 242 and suction ports 230 should have relative sizes allowing for a margin of body tissue to be located laterally therebetween within the suction ports 230.

In operation, the proximal ends 106 and 112 of the first and second legs 102 and 108, respectively, are movably connected by the guide track 118 and the turnbuckle assembly 120. The turnbuckle assembly 120 is adapted to provide lateral tension to the body tissue engaged by the suction pads 128 as follows.

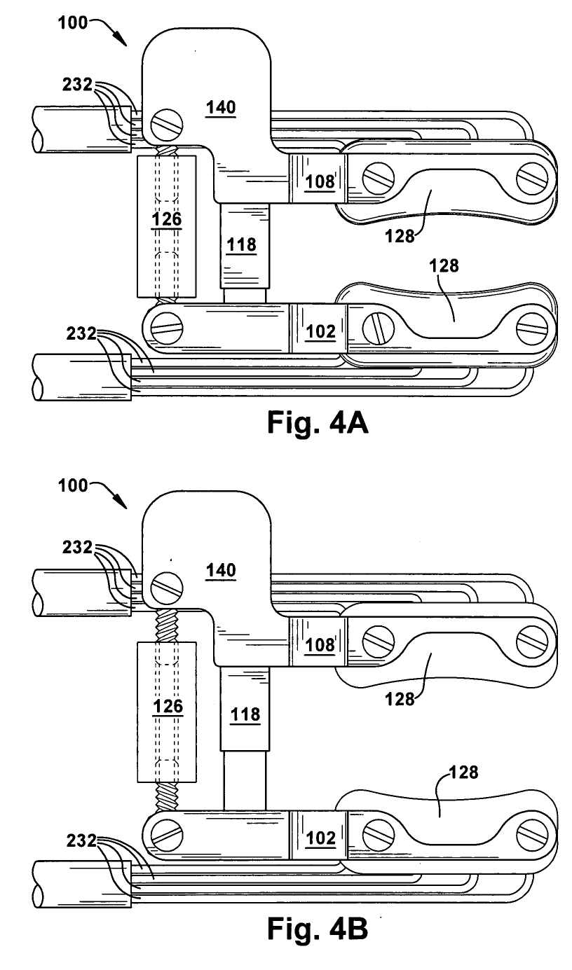

The sequence of operation of the stabilization device 100 is shown in FIGS. 4A-4B. In FIG. 4A, the stabilization device 100 is depicted in a first condition, wherein the first and second legs 102 and 108 are relatively near to each other. The turnbuckle assembly 120 is then manipulated to provide relative motion between the first and second legs 102 and 108.

In the embodiment shown in FIGS. 4A-4B, this occurs by a user applying force longitudinally to rotate the turnbuckle body 126 in a chosen direction. As the turnbuckle body 126 is rotated, the first and second threaded posts 122 and 124 are driven toward or away from each other laterally, depending upon the direction of rotation, thus moving the first and second legs 102 and 108 laterally toward or away from each other. The thread characteristics and materials of the first and second threaded posts 122 and 124 and of the turnbuckle body 126 should be chosen to be self-locking—i.e., so that engagement of the turnbuckle body with the first and second threaded posts prevents lateral forces transmitted through the first and second threaded posts from changing the positions of the first and second legs 102 and 108 without user intervention.

The guide track 118 extends or contracts, driven by the turnbuckle assembly 120, to guide the motion of the stabilization device 100 and help ensure that the movement of the first and second legs 102 and 108 is substantially lateral. The guide track 118 also helps to absorb any pivoting forces produced by lateral movement of the first and second legs 102 and 108 when the suction ports 230 resist lateral movement due to engagement with the heart tissue. Since the guide track 118 absorbs forces produced normal to the heart tissue, the turnbuckle assembly 120 can operate smoothly and easily through an entire range of motion without deleterious forces being transmitted from the heart tissue.

At least one of the suction ports 230 can be engaged with the heart tissue when the first and second legs 102 and 108 are at any stage or position in their relative lateral movement. To engage the heart tissue, the stabilization device 100 is positioned so that at least one suction pad 128 contacts the heart tissue, with one of the first and second legs 102 and 108 located at either side of a desired surgical site, to partially frame the heart tissue sought to be stabilized. The vacuum source 234 is optionally actuated to provide suction to the stabilization device 100 either before or after the suction pads 128 contact the heart tissue. Once the suction pads 128 are in contact with the heart tissue and the vacuum source 234 is actuated, one or more of the suction ports 230 engage and hold the heart tissue through suction power.

While the heart tissue is held by at least one suction port 230, the first and second legs 102 and 108 may be moved laterally to tension the heart tissue held by the suction ports 230. For example, the turnbuckle assembly 120 could be actuated to move the first and second legs 102 and 108 further apart and smooth/spread the heart tissue between the suction pads 128 into a desired stabilized position using lateral tension. Alternately, the turnbuckle assembly 120 could be actuated to move the first and second legs 102 and 108 closer together to stabilize the heart tissue located adjacent, but not between, the suction pads 128 or to return the heart tissue to an original position and thus prevent “spring-back” when the stabilization device 100 is removed. The heart tissue held by the stabilization device 100 may be moved into a desired orientation by manipulation of the stabilizing arm, with the motion of the stabilizing arm transmitted to the stabilization device 100 by the mounting plate 140 and any other mounting structures.

To disengage the stabilization device 100 from the heart tissue, either for repositioning or when stabilization is no longer desired, the vacuum pressure from the vacuum source 234 is interrupted. The suction ports 230 will automatically release the held heart tissue upon such cessation of suction power.

While aspects of the present invention have been particularly shown and described with reference to the preferred embodiment above, it will be understood by those of ordinary skill in the art that various additional embodiments may be contemplated without departing from the spirit and scope of the present invention. For example, multiple legs and corresponding turnbuckle assemblies could be provided. The turnbuckle assembly and/or guide track could be located in a center portion of the first and second legs, with suction pads provided on both the distal and proximal ends of the first and second legs. The mounting plate could instead be a pin, socket, or other structure adapted to engage a stabilizing arm. The components of the stabilization device could be made of any suitable materials, using any suitable manufacturing techniques. At least one of the legs could include a “dogleg” structure to laterally offset the suction pad from the turnbuckle assembly, with legs having mirror-image doglegs at least partially framing a polygonal area of heart tissue. A device or method incorporating any of these features should be understood to fall under the scope of the present invention as determined based upon the claims below and any equivalents thereof.

The method and apparatus of certain embodiments of the present invention, when compared with other apparatus and methods, may have the advantages of: resisting destabilizing forces produced by a beating heart in multiple orientations, being easily adjusted by a user, avoiding unwanted dislodgement from the heart surface, using existing vacuum sources, being usable in a timely and efficient manner, and being more economical to manufacture and use. Such advantages are particularly worthy of incorporating into the design, manufacture, and operation of stabilization devices. In addition, the present invention may provide other advantages which have not yet been discovered.

Other aspects, objects, and advantages of the present invention can be obtained from a study of the drawings, the disclosure, and the appended claims.

Having described the invention, the following is claimed:

1. An apparatus for holding a body tissue in a desired orientation, the apparatus comprising:
a first leg having longitudinally spaced distal and proximal ends;
a second leg, laterally spaced from the first leg, adapted for lateral motion with respect to the first leg, and having longitudinally spaced distal and proximal ends;
the distal ends of the first and second legs for contacting the body tissue;
the first leg including a first guide post extending toward the second leg, the second leg including a second guide post extending toward the first leg, the first and second guide posts telescopically engaging to form a guide track;
a first threaded post extending from the first leg toward the second leg;
a second threaded post extending from the second leg toward the first leg;
a turnbuckle body connecting the first and second threaded posts to form a turnbuckle assembly;
the proximal ends of the first and second legs being movably connected by the guide track and the turnbuckle assembly; and
the distal ends of the first and second legs each including a suction pad adapted to apply suction pressure to the body tissue to hold the body tissue in the desired orientation.

2. The apparatus of claim 1, wherein each suction pad includes one or more suction ports, and a plurality of suction tubes connect the suction ports in fluid communication with a vacuum source.

3. The apparatus of claim 2, wherein each suction tube connects one suction port with the vacuum source, and each suction tube provides independent suction control to an associated suction port.

4. The apparatus of claim 1, wherein each suction pad includes one or more suction ports, each suction port includes a suction cup, and a plurality of suction tubes connect the suction cups in fluid communication with a vacuum source.

5. The apparatus of claim 1, wherein the turnbuckle body is smaller in a longitudinal dimension than in a lateral dimension.

6. The apparatus of claim 1, wherein the distal ends of the first and second legs each include a suction pad adapted to engage body tissue using suction pressure, and the turnbuckle assembly is adapted to provide lateral tension to the body tissue engaged by the suction pads.

7. The apparatus of claim 1, wherein one of the first and second legs includes a mounting plate adapted to engage with a stabilizing arm and hold the body tissue in a desired orientation with respect to the stabilizing arm.

8. A method of holding a body tissue in a desired orientation, the method comprising the steps of:
providing a stabilization device including a first leg having longitudinally spaced distal and proximal ends and a second leg, laterally spaced from the first leg, adapted for lateral motion with respect to the first leg and having longitudinally spaced distal and proximal ends;
movably connecting the proximal ends of the first and second legs;
providing each of the distal ends of the first and second legs with a suction pad adapted to apply suction pressure to the body tissue and including a plurality of suction ports;
placing each of the plurality of suction ports into separate fluid communication with a vacuum source;
supporting the stabilization device with a stabilizing arm;
contacting the body tissue with at least one suction pad; and
holding the body tissue in a desired orientation with the stabilizing arm through suction provided from the vacuum source individually to each of the plurality of suction ports.

9. The method of claim 8, wherein the stabilization device includes a turnbuckle assembly and a guide track extending between the first and second legs, and wherein the method includes the steps of:
manipulating the turnbuckle assembly to provide relative lateral motion between the first and second legs;
guiding the relative lateral motion with the guide track; and
tensioning the body tissue through movement of the suction pads by the first and second legs while the body tissue is held by at least one suction port.
